(19) United States
(12) Patent Application Publication
(10) Pub. No.: US 2008/0153791 A1
(43) Pub. Date: Jun. 26, 2008

(54) 11Beta -Hydroxysteroid Dehydrogenases

(75) Inventor: Thomas Wilckens, Munchen (DE)

Correspondence Address: ROTHWELL, FIGG, ERNST & MANBECK, P.C., 1425 K STREET, N.W., SUITE 800, WASHINGTON, DC, 20005, US

(73) Assignee: onpharm GmbH, Wien (CH)

(21) Appl. No.: 11/908,903

(22) PCT Filed: Mar. 20, 2006
(86) PCT No.: PCT/EP2006/002538

§ 371 Date: Sep. 17, 2007

(30) Foreign Application Priority Data
Mar. 18, 2005 (EP) 05006040.9

(51) Int. Cl. A61K 31/56; A61K 31/19; A61P 3/00; A61P 3/10; A61P 27/06; A61P 5/00; A61P 35/00; A61P 3/04; A61K 31/215

(52) U.S. Cl. 514/178; 514/563; 514/529

(57) ABSTRACT

The present invention relates to novel 11β-HSD inhibitors as well as to the use of 11 β-HSD inhibitors for the manufacture of pharmaceutical agents for the prevention and/or treatment of metabolic diseases, cancer, cell proliferation, glaucoma, diseases associated with abnormal growth hormone secretion as well as wound healing disorders.

The present invention relates to novel 11β-HSD inhibitors as well as to the use of 11β-HSD inhibitors for the manufacture of pharmaceutical agents for the prevention and/or treatment of metabolic diseases, cancer, cell proliferation, glaucoma, diseases associated with abnormal growth hormone secretion as well as wound healing disorders.

### BACKGROUND OF THE INVENTION

11β-Hydroxysteroid dehydrogenase (11β-HSD) is an enzyme system that catalyses the interconversion of active glucocorticoids to their inactive metabolites, and is now established as a crucial mechanism modulating corticosteroid hormone action. Two isozymes have been identified. In vivo, 11β-HSD1 acts predominantly as an oxoreductase using NADP(H) as a cofactor to generate cortisol, whereas 11β-HSD2 acts exclusively as an NAD-dependent dehydrogenase, inactivating cortisol to cortisone. Alterations in its activity have been implicated in several human diseases, including hypertension, intra-uterine growth retardation and obesity. With the ever-increasing interest in 11β-HSD, there have also been several new tissue types and disease processes in which this enzyme system has been identified. The cellular actions of corticosteroid hormones are largely mediated through binding to nuclear receptors that act as ligand-inducible transcription factors. In mammalian tissues, the two isozymes of 11β-hydroxysteroid dehydrogenase (11β-HSD) have a pivotal role in the prereceptor regulation of corticosteroid hormone action [1], catalysing the interconversion of hormonally active glucocorticoids (GCs; cortisol and corticosterone) and their inactive 11-keto forms (cortisone and 11-dehydrocorticosterone).

11β-HSD1 was originally isolated from rat liver [2], and the gene, which is located on chromosome 1q32.2, includes six exons, is over 30 kb in length [3] (largely attributed to the length of intron 4 (25 kb)) and encodes a 34-kDa protein that resides within the endoplasmic reticulum. 11β-HSD1 enzyme activity is bidirectional, possessing both dehydrogenase (cortisol to cortisone) and reductase (cortisone to cortisol) components [1]. It is predominantly a reductase when studied in intact cells or organs in vivo, as has been shown in liver and adipose tissue [4], whereas in tissue homogenates and upon purification, dehydrogenase activity prevails [5,6]. However, under certain conditions such as reduced co-factor availability and/or in disease states its dehydrogenase (cortisol inactivation) might predominate.

By contrast, the human 11β-HSD2 isozyme is a high-affinity NAD-dependent, unidirectional dehydrogenase that converts cortisol to cortisone [1]. The gene is located on chromosome 16q22, is 6.2 kb in length, comprising five exons [7] and encoding a 44-kDa protein, which shares only 14% sequence homology with 11β-HSD1. Our understanding of the function of 11β-HSD2 has uncovered an important physiological observation. Mineralocorticoid receptors (MRs) have equal affinity for aldosterone and cortisol, and 11β-HSD2 functions to protect the MR from illicit occupation by the higher circulating concentrations of cortisol, and its consequent inactivation to cortisone [8]. Therefore, tissue distribution is generally restricted to mineralocorticoid target tissues, such as kidney [9], sweat glands, salivary glands and colonic mucosa [10], where it colocalizes with the MR and is intricately involved in salt and water balance. Genetic defects in each isozyme have been associated with human disease. Mutations in the gene encoding 11β-HSD2 (HSD 11β) give rise to a rare, inherited form of hypertension, the syndrome of apparent mineralocorticoid excess (AME) [11]. In patients with this disease, in spite of normal circulating concentrations, cortisol induces mineralocorticoid hypertension. AME represents a spectrum of diseases with a close correlation between genotype and phenotype. ‘Severe’ mutations, as characterized by a lack of conversion of cortisol to cortisone when mutant cDNAs are expressed In vitro [12], result in grossly raised urinary cortisol:cortisone metabolite ratios and juvenile- or neonatal-onset hypertension. Conversely, and of relevance to broader populations of hypertensives, patients with ‘milder’ mutations present with an intermediate biochemical phenotype (i.e. less abnormal urinary cortisol:cortisone metabolite ratios) in adolescence or early adulthood [13]. Heterozygotes often present with low-renin ‘essential’ hypertension in late adult life, notably both parents of the original index case of AME described by Edwards and Stewart [8]. Apparent cortisone reductase deficiency, in many ways the exact opposite of AME, has been reported in several anovulatory, hyperandrogenic women and patients with polycystic ovary syndrome (PCOS). Patients display a deficiency in the conversion of cortisone to cortisol that results in an increased metabolic clearance rate of cortisol. This is the stimulus to activate the hypothalamus- pituitary-adrenal axis to maintain normal circulating cortisol concentrations, but at the expense of adrenocorticotrophin- mediated adrenal androgen excess. As a consequence, patients show hirsutism, acne and oligo- or amenorrhea [14-17]. The biochemical abnormalities suggest a defect in the gene encoding 11β-HSD1 (HSD 11β) but, to date, no mutations have been identified, although only exonic regions have been sequenced and in only a few cases.

The ability of peripheral tissues to regulate corticosteroid concentrations through 11β-HSD isozymes is established as an important mechanism in the pathogenesis of diverse human diseases. Modulation of enzyme activity offers a novel therapeutic approach to treating human disease by circumventing the consequences of systemic GC excess or deficiency. However, to achieve this goal, the development of specific inhibitors that do not interfere with other enzymes is required.

The present invention describes the generation of specific inhibitors against 11β-HSD1 and/or 11β-HSD2 to either selectively or combined inhibition of the enzymes. Furthermore, the inhibitors allow the tissue specific fine tuning of local cortisol levels to compensate for cortisol excess or deficiencies.

It was an object of the invention to provide 11β-HSD inhibitors for use in other fields of application. According to the invention this object is achieved by the use of 11β-HSD inhibitors for the production of a pharmaceutical agent for the prevention and/or treatment of metabolic diseases or cancer and/or cell proliferation or glaucoma or of diseases associated with abnormal growth hormone secretion or of wound healing disorders. The 11β-HSD inhibitors used according to the invention preferably are an 11β-HSD inhibitor or a salt thereof, wherein said 11β-HSD type 1 inhibitor is selected from the group consisting of the following formulas I to III:

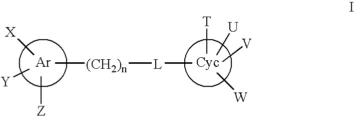

wherein
X, Y and Z each independently represent halogen, in particular, F, Cl, I or Br, C₁-C₆ alkyl, C₅-C₁₅ aryl or C₁-C₆ alkoxy,
n represents an integer from 1 to 10, in particular, from 1 to 4,
L represents an amide, amine, sulfonamide, ester, thioester or keto group,
T, U, V and W each independently represent an oxo, thio, ketone, thioketone, C₁-C₆ alkyl or C₁-C₆ alkanol group,
Ar represents an aromatic ring system, and
Cyc represents a cyclic ring system,

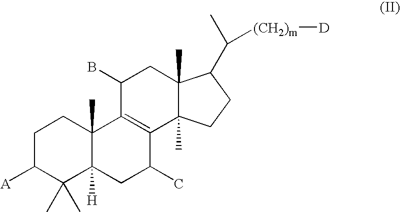

wherein
A represents a C₁-C₁₀ ester (C₁-C₁₀ alkyl-CO—O—), a C₁-C₁₀ amide (C₁-C₁₀ alkyl-CO—NH—), a C₁-C₁₀ ether or a C₁-C₁₀ ketone (C₁-C₁₀ alkyl-CO—) group,
B and C each independently represent an oxo group, a keto group, a C₁-C₆ alkanol group or a C₁-C₆ alkyl group,
m is an integer from 1 to 10, in particular, from 1 to 4, and
D is a group selected from COOR¹ or CONR²R³, wherein R¹, R² and R³ each independently represent H or a C₁-C₆ alkyl group,

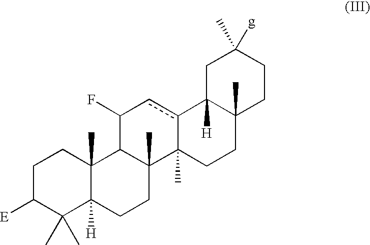

wherein
E represents an OH, a C₁-C₁₀ ester (C₁-C₁₀ alkyl-CO—O—), a C₁-C₁₀ amide (C₁-C₁₀ alkyl-CO—NH—), a C₁-C₁₀ ether (C₁-C₁₀—O—) or a C₁-C₁₀ ketone (C₁-C₁₀ alkyl-CO—) group,
F represents an oxo group, keto group, a C₁-C₆ alkanol group or a C₁-C₆ alkyl group, and
G is group selected from COOR¹ or CONR²R³, wherein R¹, R² and R³ each independently represent H or a C₁-C₂₀ hydrocarbon group, in particular, a C₁-C₆ alkyl group.

In a special embodiment, the 11β-HSD inhibitor used is 18-β-glycyrrhetinic acid or a derivative thereof such as glycyrrhizine, glycyrrhizinic acid, carbenoxolone or 2-hydroxyethyl-18β-glycyrrhetinic acid amide.

The inhibitors used according to the invention are inhibitors of 11β-HSD-type 1 and/or type 2. Particularly preferably, selective 11β-HSD-type 1 inhibitors or selective 11β-HSD-type 2 inhibitors are concerned. Especially preferably, one of the compounds substance 1 to substance 29 are concerned, even more preferably, one of the compounds selected from substance 16, substance 7, substance 13, substance 24, substance 25, substance 9 or substance 14, and most preferably, substance 16 or substance 7. It has been found that, in particular, compounds 16 and 7 are selective 11β-HSD1 inhibitors which show no inhibition for 11β-HSD2, 17β-HSD1, 17β-HSD2.

It has also been found that the measured inhibition behavior of compounds can be different when using either cell lysates or intact cells. This means that, in particular, data obtained from cell lysates or other in vitro systems cannot reflect the effect of the substances in vivo. For example, especially compounds based on glycyrrhetinic acid, in particular, compounds having formula III as described herein, often show higher inhibition of 11β-HSD2, compared with 11β-HSD1, in analyses using cell lysates. When using intact cells as a measurement system, however, a switch in their preference is observed to inhibit 11β-HSD1 instead of 11β-HSD2. However, inhibition of 11β-HSD1 has considerable therapeutic use for glucocorticoid-associated diseases including obesity, diabetes, wound healing and muscle atrophy. Since inhibition of related enzymes such as 11β-HSD2 and 17β-HSDs cause sodium retention and hypertension or interfere with 6-steroid hormone metabolism, highly selective 11β-HSD1 inhibitors are required for successful therapy. Herein, several selective inhibitors and medical applications thereof are presented. For example, compound 16 shows an IC₅₀ [μM] for 11β-HSD1 of 0.144±0.27 determined in lysates of HEK-293 cells and an IC₅₀ [μM] for 11β-HSD1 of 0.41±0.08 determined in intact transfected HEK-293 cells and, thus, a high specificity for this enzyme while it shows IC₅₀ [μM] values for 11β-HSD2 (determined in lysates of HEK-293 cells) of 3.95±0.12, for 17β-HSD1 of greater than 30 μM, for 17β-HSD2 of 28.3±5.5 and for 11β-HSD2 (determined in intact transfected HEK-293 cells) of greater than 50 μM.

### 11β-HSD1, Obesity and Insulin Sensitivity

One aspect of the present invention, therefore, is the use of an 11β-HSD inhibitor or a pharmaceutically acceptable salt thereof, in particular, selected from the group consisting of substances 7, 13, 14, 16, 24, 25, 1-6, 8-12, 15, 17-23 or a compound of formula I, II or III for the manufacture of a pharmaceutical agent for the prevention and/or treatment of metabolic diseases. For this medical indication, preferably 11β-HSD1 inhibitors are used.

Specifically, the invention relates to the prevention and/or treatment of obesity or insulin sensitivity and, in particular, diabetes type II.

In patients with Cushing's syndrome, florid but reversible central adiposity is observed in the setting of GC excess. This observation stimulated several groups to assess the involvement of cortisol in the development of visceral adiposity. However, in simple obesity, circulating cortisol concentrations are normal (occasionally lower), with increased cortisol secretion rates [35], suggesting that, in peripheral tissues, cortisol metabolism via 11β-HSDs, might be disturbed. 11β-HSD1, but not 11β-HSD2, is synthesized in human adipose tissue, both in preadipocytes and adipocytes. In vitro studies confirmed the stimulatory effects of cortisol on the differentiation of adipose stromal cells to mature adipocytes, with inhibition of 11β-HSD1 by glycyrrhetinic acid preventing cortisone-induced differentiation [36]. This work has been extended with the development of in vivo transgenic animal models. Overexpression of HSD 11β1 in adipose tissue resulted in a threefold accumulation of visceral adipose tissue [37]. Crucially, this work showed that prereceptor metabolism of GCs by 11β-HSD1 resulted in increased ‘active’ GC concentrations within adipose tissue and represents an important step forward. Most clinical studies [38,39], but not all [40], have shown decreased 11β-HSD1 activity in obesity by measuring urinary cortisol:cortisone metabolite ratios. However, such studies have mainly measured hepatic 11β-HSD1 activity and it has been widely hypothesized that, within adipose tissue itself, there is an increase in 11β-HSD1 synthesis in obesity. This pattern of expression has been shown in the rodent model of obesity, the Zucker rat [41], but there have been conflicting reports from human studies. Data on subcutaneous abdominal biopsies from obese men and women showed higher levels of enzyme activity in cell homogenates in two separate studies [39,40]. Furthermore, mRNA analyses have shown increased 11β-HSD1 synthesis in obese subcutaneous adipocytes of obese people. However, using paired omental and subcutaneous samples from over 30 patients, Tomlinson et al. [42] were unable to correlate whole-tissue or adipocyte 11β-HSD1 synthesis with obesity. Crucially, however, they did show a mild reduction in synthesis and activity from cultured preadipocytes in obese individuals. This could lead to enhancement of proliferation of the preadipocytes owing to decreased cortisol generation. As a consequence, more preadipocytes will be available to undergo differentiation and lipid accumulation, and in this way could contribute to the increases in visceral adipose tissue mass seen in obese patients. In summary, it seems unlikely that over expression of 11β-HSD1 in visceral adipose tissue is a primary cause of obesity, but inhibition of the autocrine generation of cortisol at this site might lead to reduced adipogenesis.

Further compelling data that 11β-HSD1 can increase intracellular GC concentrations have come from studies of liver, in which GCs oppose the actions of insulin by regulating key gluconeogenic enzymes. Pharmacological inhibition of 11β-HSD1 in healthy men [43] and patients with type 2 diabetes mellitus [44] caused a lowering of intrahepatic GC levels, leading to reduced hepatic glucose output and enhanced lipid catabolism. Mice with targeted disruption of HSD 11β, resistant to stress-induced or high-fat diet-induced obesity, showed increased insulin sensitivity [45] and enhanced lipid oxidation [46]. Recently, a new selective class of 11β-HSD1 inhibitor, the arylsulfonamidothiazoles [47], has been developed which, in a mouse model, has been shown to improve insulin sensitivity [48], and which could therefore be useful for the treatment of diabetes mellitus.

### Cancer and Cell Proliferation

Another object of the present invention is the use of the 11β-HSD inhibitors for the manufacture of a pharmaceutical agent for the prevention and/or treatment of cancer and/or cell proliferation. The compounds described herein are excellently suited, in particular, for the treatment of breast cancer, colon cancer, leukemia or gastrointestinal cancer. For these medical indications, preferably 11β-HSD2 inhibitors are used.

Based on these precedents, there has been great interest in the role of 11-HSD2 in human hypertension and, to a lesser extent, the role of 11β-HSD1 in PCOS [18]. However, as we have acquired more knowledge about the expression, function and regulation of 11β-HSD isozymes, other unexpected connections have emerged. Thus, we could establish a relationship between 11β-HSD2 and cancer. An established role of GCs is to inhibit cell proliferation and stimulate cellular differentiation. Although 11β-HSD1 and 11β-HSD2 are the products of separate genes and are synthesized in distinct tissues, the concept that their actions are entirely separate no longer appears to be true. Instead, it seems that, at key points in development, there is a ‘switch’ occurring between the anti-proliferative effects of 11β-HSD1 and the pro-proliferative effects of 11β-HSD2. In contrast to normal tissues, 11β-HSD2 in this context appears to regulate cortisol exposure to the GC receptor (GR), rather than to the MR, and evidence for this has come from studies of normal tissues throughout development. Throughout early foetal development, 11β-HSD2 is synthezised in many tissues, such as bone and adrenal, where, in adult life, 11β-HSD1 is synthesized [19]. A more dramatic switch in 11β-HSD synthesis implicates 11β-HSD2 as a putative oncogene. In several neoplastic cells types, there is a high level of expression of HSD 11β2 in contrast to normal tissue equivalents, which only synthesize 11β-HSD1. For example, adrenal cortical adenomas and carcinomas synthesize 11β-HSD2 [20]. The relative synthesis of 11β-HSD2 can be used to determine the phenotype of adrenal adenomas, with high levels of mRNA in non-functioning adenomas and adenomas causing overt Cushing's syndrome [21]. In the pituitary, there is a marked difference between normal and tumourous tissue with respect to 11β-HSD2 synthesis. 11β-HSD2 levels are high in pituitary tumours, irrespective of type, and the enzyme is virtually absent from normal tissue [22]. Quantitative real-time PCR revealed a change in isozyme synthesis from 11β-HSD1 in normal pituitaries to a tenfold induction of the synthesis of 11β-HSD2 in tumours [23]. Rabbitt and co-workers further investigated the role of 1-HSDs in cellular proliferation, using transfection experiments [24]. In stably transfected cells overexpressing HSD 11β2, cellular proliferation was increased compared with mock-transfected cells; conversely, proliferation rates were lower in cells overexpressing HSD 11β1.

11β-HSD2 synthesis has also been documented in ductal and lobular breast epithelial cells [25], with increased synthesis of 11β-HSD2 observed in invasive carcinomas. This, taken together with the observation that inhibition of 11β-HSD2 potentiates the antiproliferative actions of GCs in some breast [26] and endometrial cancer cell lines [27], further endorses a putative role for 11β-HSD2 activity in tumourigenesis

### 11β-HSD1 and Glaucoma

The invention further relates to the use of the 11β-HSD inhibitors described herein for the treatment of glaucoma. For this medical indication, preferably 11β-HSD1 inhibitors are used.

Topical and systemic GCs are used in a diverse range of conditions in clinical opthalmology, and one of the most significant complications is corticosteroid-induced glaucoma. This condition is characterized by a significant increase in intraocular pressure (IOP), which, if untreated, can lead to visual field loss and blindness. IOP is maintained by a balance between production and drainage of aqueous humour. The major site of aqueous production is from the non-pigmented epithelial cells (NPE) of the ciliary body, whereas drainage is predominantly through the cells of the trabecular meshwork. The eye represents an important target tissue for corticosteroids, containing both MRs [28] and GRs [29]. Corticosteroids have long been implicated in the natural diurnal variation of IOP [30], and raised IOP can also occur in patients with Cushing's syndrome [31]. Several groups have used immunohistochemical and in situ hybridization analyses to assess the synthesis of 11β-HSDs in a variety of human ocular tissues, and have reported conflicting results. One of the studies localized HSD 11β2 mRNA and the 11β-HSD2 protein in the NPE, with coexpression of MR [32]. Because the NPE has morphological characteristics of epithelia engaged in salt and water transport, this was perhaps not surprising. However, Stokes et al. [33] and Rauz et al. [34] localized 11β-HSD1 to this tissue type, suggesting that it is this isozyme that has an important role in aqueous humour production. Rauz and co-workers also demonstrated mRNA for GR, MR and 11β-HSD1 (but not for 11β-HSD2) in a human ciliary epithelial cell line, ODM-2 [34]. In addition, they noted that aqueous humour concentrations of ‘free’ cortisol greatly exceeded those of cortisone (GC/MS analysis-cortisol:cortisone ratio 14:1, compared with circulating cortisol:cortisone of, 3:1), consistent with local 11β-HSD1 activity generating cortisol from cortisone. The functional significance of 11β-HSD1 in the eye was then investigated by administering a non-specific 11β-HSD inhibitor, carbenoxolone (CBX), to healthy volunteers [34]. After seven days of CBX, IOP was reduced by 17.5%, in keeping with the hypothesis that inhibition of 11β-HSD1 within the NPE reduces local cortisol generation, causing a fall in IOP (FIG. 3). An important application of these findings could be in the therapeutic management of glaucoma, with topical preparations of CBX or more selective 11β-HSD1 inhibitors effective in lowering IOP. However, a more critical analysis defining the role of 11β-HSD1 in regulating epithelial Nap transport within the eye and HSD 11β1 expression in glaucoma is required.

### Growth Hormone and 11β-HSD

The invention also relates to the use of the 11β-HSD inhibitors for the prevention and/or treatment of diseases associated with abnormal growth hormone secretion.

Many of the clinical features of patients with abnormal growth hormone (GH) secretion can be explained by altered 11β-HSD activity, notably hypertension in acromegaly [55,56] and obesity, insulin resistance and osteopaenia in GH deficiency (GHD) [57]. Neither GH nor insulin-like growth factor I (IGF-I) has an effect upon renal 11β-HSD2 activity, and the increased Na retention seen in acromegaly is unlikely to involve this mechanism. However, hypopituitary GHD patients have raised urinary cortisol:cortisone metabolite ratios. These return to normal upon replacement therapy with GH, and are indicative of a decrease in 11β-HSD1 oxoreductase activity [58]. Similarly, in patients with active acromegaly, there is a decrease in the cortisol:cortisone metabolite ratio that corrects with suppression of GH levels by surgery, somatostatin analogues or GH receptor antagonists [59,60], indicative of increased 11β-HSD1 activity. In vitro, IGF-I but not GH itself, inhibits 11β-HSD1 [59]. These data could have important clinical ramifications, and it is interesting to speculate that the phenotype of GH deficiency in the context of hypopituitarism (obesity, insulin resistance and osteoporosis) might be an indirect effect of GH action on cortisol metabolism through 11β-HSD1.

### Other Therapies

The invention also relates to the use of the 11β-HSD inhibitors for the treatment of wound healing disorders.

### Bone Physiology and 11-β-HSD

The adverse effects of GCs on skeletal tissue have been recognized for many years and the continued exposure of bone cells to even modest doses of GCs results in osteoporosis [49]. However, these are mainly attributable to nonphysiological treatment with synthetic corticoids, which usually exceed physiological levels by a factor of 10-100. In vivo, this is partly a result of the indirect effects of GCs on Ca2p homeostasis and synthesis of skeletal growth factors and hormones. In vitro, GCs have effects on both bone-resorbing cells (osteoclasts) and bone-forming cells (osteoblasts), where their actions are complex, involving both direct and indirect effects on proliferation and differentiation in both cell types. This has prompted much recent work to assess the relative synthesis of 11β-HSD1 and -2 in bone and their ability to influence local GC concentrations within this tissue type. Using human osteosarcoma cell lines, Bland et al. [50] first described the presence of 11β-HSD dehydrogenase activity in osteoblastic cells. This was found to correlate with GR expression levels rather than those of MR. Subsequent enzyme kinetic and mRNA analyses determined the presence of 11β-HSD2 in these osteosarcoma cells. These observations were further supported by studies using rat osteosarcoma cells [51]. By contrast, in cultures of primary human osteoblasts, and cells from normal adult bone (osteoclasts and osteoblasts), 11β-HSD1 is the predominant isozyme, with 11β-HSD2 being virtually undetectable [52]. 11β-HSD1 activity was stimulated by proinflammatory cytokines, specifically interleukin 1 and tumour necrosis factor a, whereas 11β-HSD2 was inhibited [53], suggesting that these factors might sensitize skeletal tissue to GC action and might represent one of the mechanisms contributing to inflammation-mediated periarticular osteoporosis in infants. However, it must be emphasized that physiological low dose glucocorticoid therapy has been demonstrated to have bone sparing effects in diseases such as rheumatoid arthritis. Furthermore, in an established animal model for rheumatoid arthritis it was demonstrated that blocking of 11β-HSD dehydrogenase activity results in significantly reduced inflammation and bone, as well as cartilage loss. Thus, increasing endogenous glucocortioids mimics the effects of low dose physiologic corticoisteroid treatment. These findings are in stark contrast with the above formulated speculation that enhanced endogenous glucocorticoid synthesis might contribute to inflammation and/or immune mediated bone loss. Finally, it was demonstrated that patients suffering from rheumatoid arthritis may in deed have significantly lower tissue levels of cortisol as a result of enhanced cortisol catabolism (R. Straub, personal communication); since lack of endogenous glucocorticoids enhances pathological bone loss in adjuvant induced arthritis, it is becoming increasingly clear that increasing glucocortiods with either physiological supplementation or by blocking endogenous catabolism by 11-β-HSD could result in a bone sparing effect. This has recently been demonstrated by the use of an established inhibitor of cortisol catabolism; i.e. glycyrrhetinic acid. Thus, selective and more potent inhibitors to block glucocorticoid catabolism will significantly improve the treatment of pathologies associated with inflammation and/or immune mediated bone loss.

Changes in 11β-HSD1 in primary cultures of human bone have also been correlated with age, with enhanced activity reported in osteoblasts from older individuals [54], implicating a role for 11β-HSD1 in the pathogenesis of senile osteoporosis.

It is also to be understood that the compound/composition of the present invention has other important medical implications.

For example, the compound or composition of the present invention may be useful in the treatment of the disorders listed in WO-A-99/52890.

In addition, or in the alternative, the compound or composition of the present invention are useful in the treatment of the disorders listed in WO-A-98/05635. For ease of reference, part of that list is now provided: cancer, inflammation or inflammatory disease, dermatological disorders, fever, cardiovascular effects, hemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the compound or composition of the present invention are useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bane, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cells types to sites of injury or infection); haemostatic and thrombolytic activity (e.g. tor treating haemophilia and stroke); antiinflammatory activity (for treating e.g. septic shock. or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the composition of the present invention may be useful in the treatment of disorders listed in WO-A-98/09985. For ease of reference, part of that list is now provided: macrophage inhibitory and/or T cell inhibitory activity and thus, anti- inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentos; immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complications and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre Syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bane marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation or natural or artificial cells, tissue and organs such as cornea, bone marrow, organs, tenses, pacemakers, natural or artificial skin tissue.

It is particularly preferred in the above uses to employ a compound selected from substances 7, 13, 14, 16, 24 and 25.

### Pharmaceutical Compositions

In one aspect, the present invention provides a pharmaceutical composition, which comprises a compound according to the present invention and optionally a pharmaceutical acceptable carrier, diluent or excipient (including combinations thereof).

The pharmaceutical composition comprises, as an active ingredient, an 11β-HSD inhibitor or a salt thereof, wherein said 11β-HSD type 1 inhibitor is selected from the group consisting of the following formulas I to III:

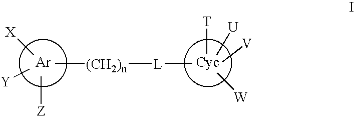

wherein
X, Y and Z each independently represent halogen, in particular, F, Cl, I or Br, C₁-C₆ alkyl, C₅-C₁₅ aryl or C₁-C₆ alkoxy,
n represents an integer from 1 to 10, in particular, from 1 to 4,
L represents an amide, amine, sulfonamide, ester, thioester or keto group,
T, U, V and W each independently represent an oxo, thio, ketone, thioketone, C₁-C₆ alkyl or C₁-C₆ alkanol group,
Ar represents an aromatic ring system, and
Cyc represents a cyclic ring system,
with the proviso that the compound is not substance 7,

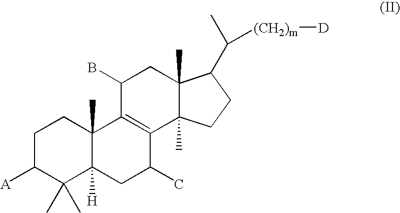

wherein
A represents —OH, a C₁-C₁₀ ester (C₁-C₁₀ alkyl-CO—O—), a C₁-C₁₀ amide (C₁-C₁₀ alkyl-CO—NH—), a C₁-C₁₀ ether or a C₁-C₁₀ ketone (C₁-C₁₀ alkyl-CO—) group,
B and C each independently represent an oxo group, a keto group, a C₁-C₆ alkanol group or a C₁-C₆ alkyl group,
m is an integer from 1 to 10, in particular, from 1 to 4, and
D is a group selected from COOR¹ or CONR²R³, wherein R¹, R² and R³ each independently represent H or a C₁-C₆ alkyl group,
with the proviso that the compound is not substance 16,

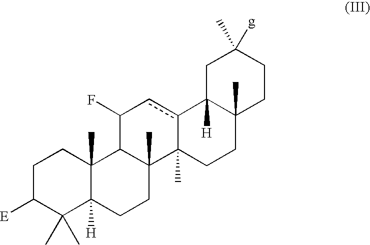

wherein
E represents an OH, C₁-C₁₀ ester (C₁-C₁₀ alkyl-CO—O—), a C₁-C₁₀ amide (C₁-C₁₀ alkyl-CO—NH—), a C₁-C₁₀ ether (C₁-C₁₀—O—) or a C₁-C₁₀ ketone (C₁-C₁₀ alkyl-CO—) group,
F represents an oxo group, keto group, a C₁-C₆ alkanol group or a C₁-C₆ alkyl group, and
G is a group selected from COOR¹ or CONR²R³, wherein R¹, R² and
R³ each independently represent H or a C₁-C₂₀ hydrocarbon group, in particular, a C₁-C₆ alkyl group,
with the proviso that the compound is not a compound of substance 24 or 25.

The pharmaceutical composition may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent.(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or preenterally in which the composition is formulated in an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit through the gastrointestinal tract; for example, it should be resistant to proteolytic degradation, stable at an acidic pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients or in the form of elixirs, solutions or suspension containing flavouring or colouring agents, or they can be injected parenterally for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Preferably, the 11β-HSD inhibitors used according to the invention are employed in the form of liposomes, oily emulsions or nanoparticles. Administration is preferably effected either i.v. or subcutaneously in the case of liposomes as well as i.m. or subcutaneously in the case of oily emulsions. When administered orally, as also preferred, the active agents are preferably provided in capsules.

### Combination Pharmaceutical

The compound of the present invention may be used in combination with one or more other active agents, such as one or more other pharmaceutically active agents.

By way of example, the compounds of the present invention may be used in combination with other 11β-HSD inhibitors.

### Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The compositions of the present invention may be administered by direct injection. The composition may be formulated for parenteral, mucosal, intramuscular, intravenous, subcutaneous, intraocular or transdermal administration. Depending upon the need, the agent may be administered at a dose of from 0.01 to 30 mg/kg body weight, such as from 0.1 to 10 mg/kg, more preferably from 0.1 to 1 mg/kg body weight.

By way of further example, the agents of the present invention may be administered in accordance with a regimen of 1 to 4 times per day, preferably once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Aside from the typical modes of delivery—indicated above—the term “administered” also includes delivery by techniques such as lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical. or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular or subcutaneous route.

Thus, for pharmaceutical administration, the 11β-HSD inhibitors of the present invention can be formulated in any suitable manner utilising conventional pharmaceutical formulating techniques and pharmaceutical carriers, adjuvants, excipients, diluents etc. and usually for parenteral administration. Approximate effective dose rates may be in the range from 1 to 1000 mg/day, such as from 10 to 900 mg/day or even from 100 to 800 mg/day depending on the individual activities of the compounds in question and for a patient of average (70 kg) body weight. More usual dosage rates for the preferred and more active compounds will be in the range 200 to 800 mg/day, more preferably, 200 to 500 mg/day, most preferably from 200 to 250 mg/day. They may be given in single dose regimes, split dose regimes and/or in multiple dose regimes lasting over several days. For oral administration they may be formulated in tablets, capsules, solution or suspension containing tram 100 to 500 mg of compound per unit dose. Alternatively and preferably the compounds will be formulated for parenteral administration in a suitable parenterally administrable carrier and providing single daily dosage rates in the range 200 to 800 mg, preferably 200 to 500, more preferably 200 to 250 mg. Such effective daily doses will, however, vary depending on inherent activity of the active ingredient and on the bodyweight of the patient, such variations being within the skill and judgement of the physician.

The compounds of the present invention are useful in the manufacture of a medicament for revealing an endogenous glucocorticoid-like effect.

### Experimental Procedures

### Materials

Cell culture reagents were purchased from Invitrogen (Carlsbad, Calif.), [1,2,6,7-3H]-cortisone from American Radiolabeled Chemicals (St. Louis, Mo.) and [1,2,6,7-3H]-cortisol from Amersham Biosciences (General Electrics Healthcare, Piscataway, N.J.). Thin layer chromatography (TLC) plates (SIL G-25 UV254) were purchased from Macherey-Nagel, Oensingen, Switzerland.

### Assay for 11β-HSD Activity

The screening assay used to determine inhibition of 11β-HSD enzyme activity is based on the conversion of radiolabelled cortisone or cortisol in cell lysates from HEK-293 cells, stably transfected with either human 11β-HSD1 or human 11β-HSD2 (Schweizer et al. 2003, Frick et al. 2004). Cells were grown in 10 cm dishes to 80% confluence and incubated for 16 h in steroid-free medium (charcoal-treated fetal calf serum (FCS) from HyClone, Logan, Utah). Cells were rinsed once with phosphate-buffered saline (PBS), detached and centrifuged for 3 min at 150×g. The supernatant was removed and the cell pellet quick-frozen in a dry-ice ethanol bath. At the day of experiment, cell pellets were resuspended in buffer TS2 (100 mM NaCl, 1 mM EGTA, 1 mM EDTA, 1 mM MgCl₂, 250 mM sucrose, 20 mM Tris-HCl, pH 7.4), sonicated and activities determined immediately. The rate of conversion of cortisol to cortisone or the reverse reaction was determined in 96-well optical PCR reaction plates (Applied Biosystems, Foster City, Calif.) in a final volume of 22 μl, and the tubes were capped during the reaction to avoid evaporation.

### Determination of Oxidase Activity Using Cell Lysates:

Reactions were initiated by simultaneously adding 10 μl of cell lysate and 12 μl of TS2 buffer containing the appropriate concentration of the inhibitory compound to be tested, NAD⁺, 30 nCi of [1,2,6,7-³H]-cortisol and unlabeled cortisol. A final concentration of 400 μM NAD⁺ and 25 nM cortisol were used. Stock solutions of the inhibitors in methanol or DMSO were diluted in TS2 buffer to yield the appropriate concentrations, whereby the concentration of methanol or DMSO in the reactions were kept below 0.1%. Control reactions with or without 0.1% of the solvent were performed. Incubation was at 37° C. for 10 min with shaking, reactions were terminated by adding 10 μl of stop solution containing 2 mM of unlabeled cortisol and cortisone dissolved in methanol. The conversion of radiolabeled cortisol was determined by separation of cortisol and cortisone using TLC and a solvent system of 9:1 (v/v) chloroform:methanol, followed by scintillation counting. In absence of inhibitors approximately 30% of cortisol was converted to cortisone.

### Determination of Reductase Activity Using Cell Lysates:

Reactions were initiated simultaneously by adding 10 μl of cell lysate and 12 μl of TS2 buffer containing the appropriate concentration of the inhibitory compound to be tested, NADPH, 30 nCi of [1,2,6,7-³H]-cortisone and unlabeled cortisone, whereby final concentrations were 400 μM NADPH and 100 nM cortisone. Activities were determined immediately after cell disruption by measuring the conversion of radiolabeled cortisone to cortisol for 10 min.

### Determination of Activities in Intact Cells:

Human 11β-HSD1 and 11β-HSD2 activities were assessed in intact stably transfected HEK-293 cells. Per well of a 96-well plate 30,000 cells were seeded, followed by growth for 24 h in steroid-free DMEM medium. The total volume was 30 μl and contained 100 nM of radiolabeled cortisol or cortisone as substrate and the corresponding inhibitor at a final concentration between 0-200 μM. The reaction was performed in steroid-free medium in the absence of exogenous cofactor for 1 to 3 h at 37° C. The solvent was below 0.1% (DMSO). Reactions were stopped by adding unlabeled cortisol and cortisone dissolved in methanol (2 mM final). Steroids were separated by TLC and conversion determined by scintillation counting.

Enzyme kinetics were analyzed by non-linear regression using Data Analysis Toolbox (MDL Information Systems Inc.) assuming first-order rate kinetics. Data represent mean ±SD of four to five independent experiments.

For references see publications 61 and 62.

Compounds

| Compound | |
|---|---|
| Name | Structure |
| Substance 1 | 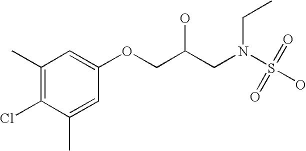 |
| Substance 2 | 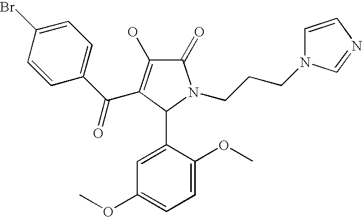 |
| Substance 3 | 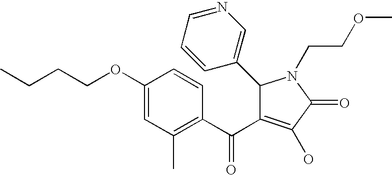 |
| Substance 4 | 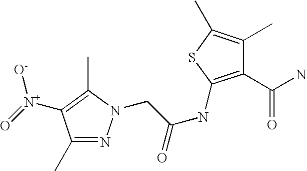 |
| Substance 5 | 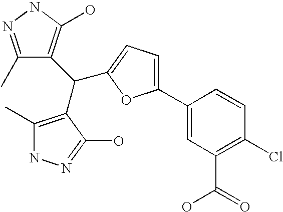 |
| Substance 6 | 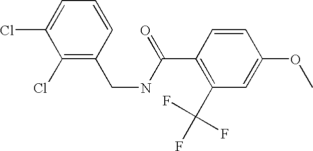 |
| Substance 7 | 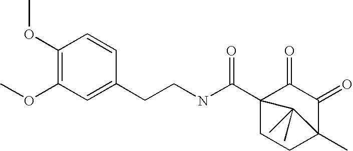 |
| Substance 8 | 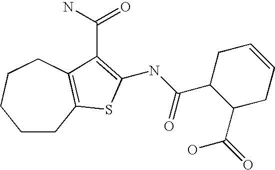 |
| Substance 9 | 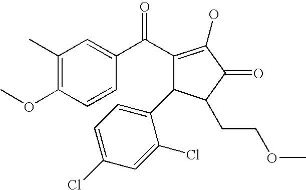 |
| Substance 10 | 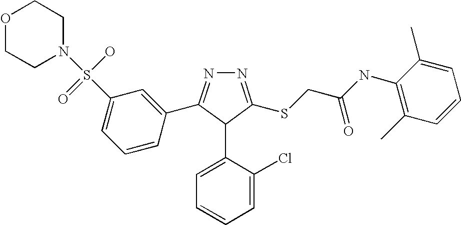 |
| Substance 11 | 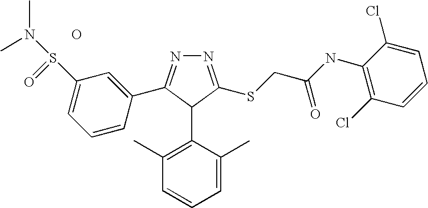 |
| Substance 12 | 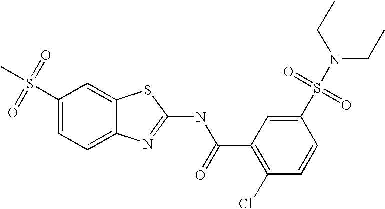 |
| Substance 13 | 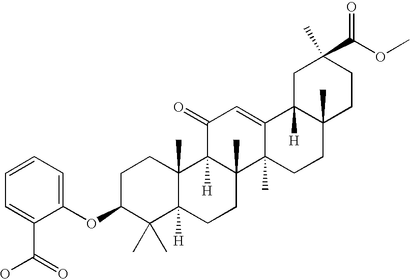 |
| Substance 14 | 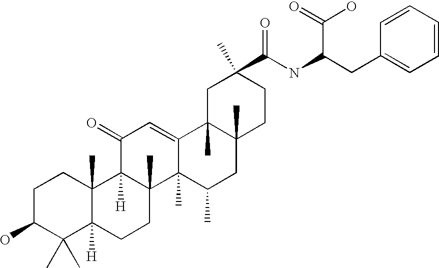 |
| Substance 15 | 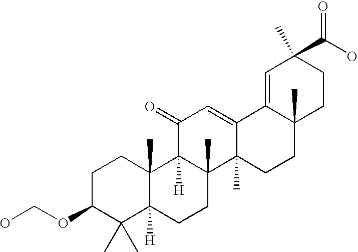 |
| Substance 16 | 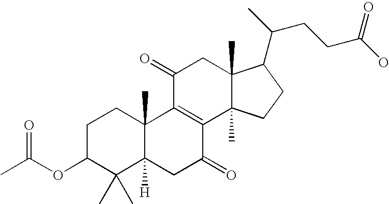 |
| Substance 17 | 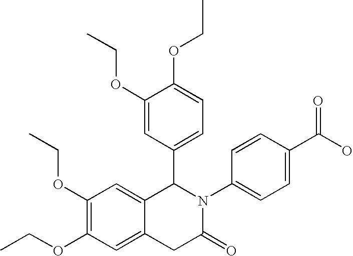 |
| Substance 18 | 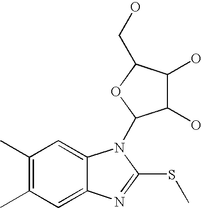 |
| Substance 19 | 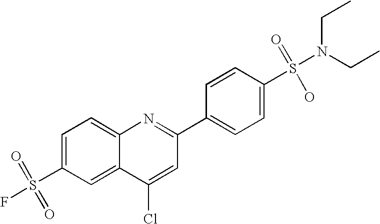 |
| Substance 20 | 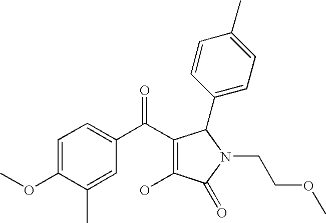 |
| Substance 21 | 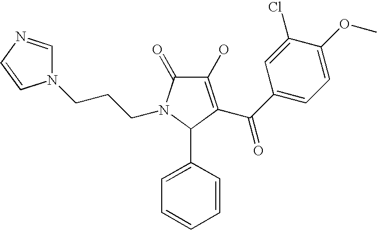 |
| Substance 22 | 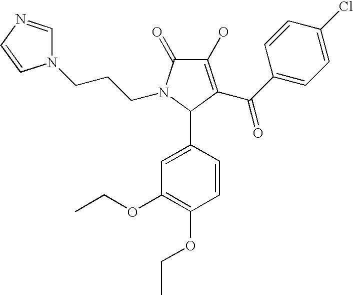 |
| Substance 23 | 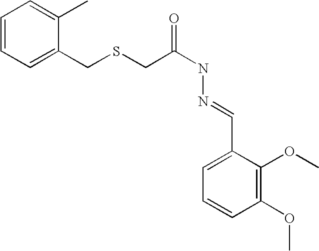 |
| Substance 24 | 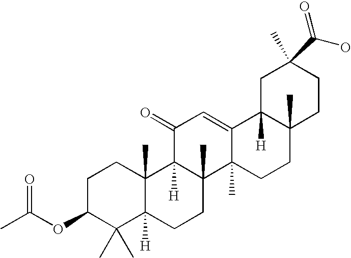 |
| Substance 25 | 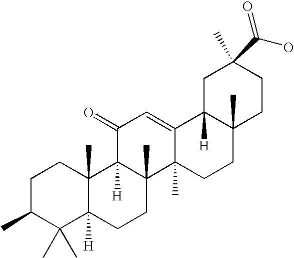 |
| Substance 26 | 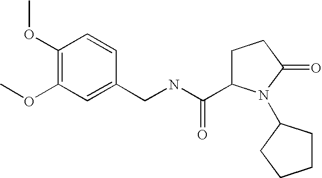 |
| Substance 27 | 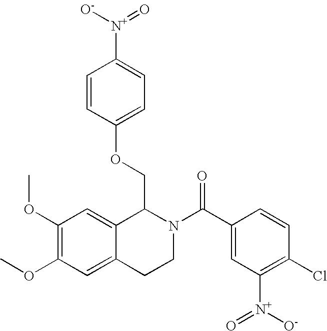 |
| Substance 28 | 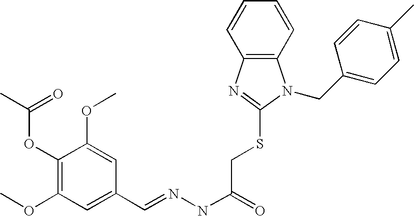 |
| Substance 29 | 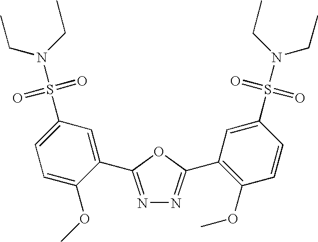 |
| Substance 30 | 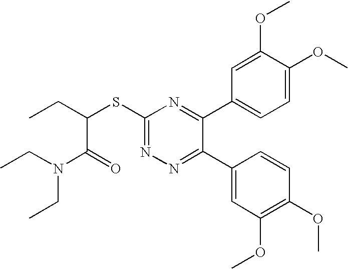 |
| Substance 31 | 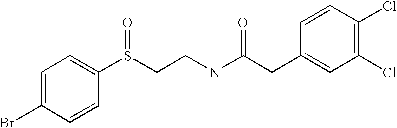 |

### Results

Inhibition of 11β-HSD1 and 11β-HSD2 in cell lysates Inhibition of 11β-HSD1 was determined at 100 nM cortisone, inhibition of 11β-HSD2 at 25 nM cortisol as substrates (at approximately 30% of apparent Km concentrations).

Assay with 20 μM of the corresponding compound in the reaction mixture, added simultaneously with the substrate:

| | 11β-HSD1% of | | 11β-HSD2% of | |
|---|---|---|---|---|
| 11β-HSD1 | control | | control | |
| control | 100 | | 100 | |
| 10 μM CBX | 4.4 | | 15.5 | |
| BNW1 | 102.1 | | 96.8 | |
| BNW2 | 78.8 | | 78.0 | |
| BNW3 | 60.3 | | 53.6 | |
| BNW4 | 82.2 | | 95.0 | |
| BNW5 | 69.8 | | 97.5 | |
| BNW6 | 79.6 | | 145.0 | |
| BNW7 | 9.6 | * | 139.5 | |
| BNW8 | 41.7 | | 102.7 | |
| BNW9 | 30.7 | | 77.4 | |
| BNW10 | 64.3 | | 128.7 | |
| BNW11 | 70.1 | | 120.9 | |
| BNW12 | 85.4 | | 132.1 | |
| BNW13 | 3.9 | * | 14.4 | * |
| BNW14 | 20.2 | * | 25.5 | * |
| BNW15 | 50.4 | | 56.9 | |
| BNW16 | 2.7 | * | 27.4 | |
| BNW17 | 88.2 | | 120.1 | |
| BNW18 | 92.0 | | 82.8 | |
| BNW19 | 51.1 | | 73.6 | |
| BNW20 | 46.8 | | 120.7 | |
| BNW21 | 48.9 | | 121.6 | |
| BNW22 | 41.3 | | 104.3 | |
| BNW23 | 85.1 | | 132.7 | |
| BNW24 | 3.9 | * | 13.3 | * |
| BNW25 | 2.9 | * | 13.9 | * |
| BNW26 | 94.1 | | 136.8 | |
| BNW27 | 78.6 | | 126.4 | |
| BNW28 | 76.7 | | 137.0 | |
| BNW29 | 75.3 | | 115.4 | |
| BNW30 | 48.4 | | 140.0 | |

Determination of IC50 values, using 7 different inhibitor concentrations at factor 2 intervals:

| | values in | | | | | | |
|---|---|---|---|---|---|---|---|
| | μM | BNW7 | BNW13 | BNW14 | BNW16 | BNW24 | BNW25 |
| 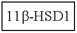 | | | | | | | |
| IC 50 | 1 | 1.95e+0 | 6.66e−1 | 2.75e+0 | 1.49e−1 | 7.33e−1 | 1.47e−1 |
| | 2 | 1.91e+0 | 7.56e−1 | 3.09e+0 | 1.68e−1 | 9.05e−1 | 2.06e−1 |
| | 3 | 2.24e+0 | 6.52e−1 | 2.58e+0 | 1.14e−1 | 7.74e−1 | 1.61e−1 |
| | Mean | 2.03e+0 | 6.91e−1 | 2.81e+0 | 1.44e−1 | 8.04e−1 | 1.72e−1 |
| | S.D. | 0.18 | 0.056 | 0.26 | 0.027 | 0.090 | 0.031 |
| 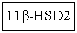 | | | | | | | |
| IC 50 | 1 | did not | out of | out of | out of | out of | out of |
| | | inhibit | range | range | range | range | range |
| | 2 | did not | 2.63e−1 | 2.01e+0 | 4.04e+0 | 1.69e−1 | 5.46e−2 |
| | | inhibit | | | | | |
| | 3 | did not | 2.99e−1 | 2.69e+0 | 3.87e+0 | 2.34e−1 | 6.49e−2 |
| | | inhibit | | | | | |
| | Mean | n.d. | 2.81e−1 | 2.35e+0 | 3.95e+0 | 2.02e−1 | 5.97e−2 |
| | S.D. | n.d. | 0.025 | 0.48 | 0.12 | 0.046 | 0.0073 |

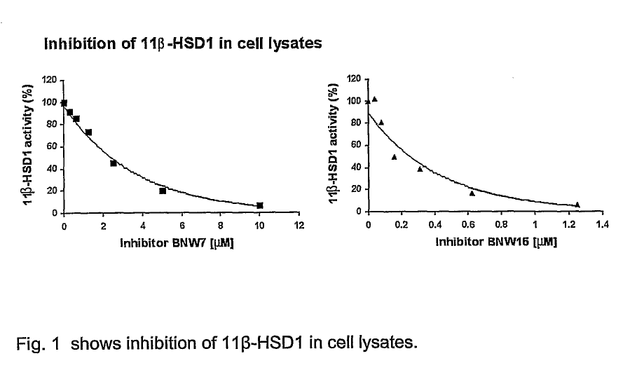

FIG. 1 shows inhibition of 11β-HSD1 in cell lysates.

### Inhibition Analysis of BNW7 and BNW16 on 11β-HSD1 Activity in Intact, Stably Transfected HEK-293 Cells

Compound BNW7 did not inhibit 11β-HSD2, 17β-HSD1 or 17β-HSD2. BNW16 inhibited 11β-HSD2 with an IC50 of 3.95 μM and 17β-HSD2 with an IC50 of 28.3 μM.

In intact HEK-293 cells transiently expressing 11β-HSD1, BNW7 is a more potent compound than BNW16, probably due to the carboxy group.

| | IC50 in | IC50 in |
|---|---|---|
| | cell lysates | intact cells |
| BNW7 | 2.03 ± 0.18 | 0.55 ± 0.05 |
| BNW16 | 0.144 ± 0.027 | 2.32 ± 0.33 |

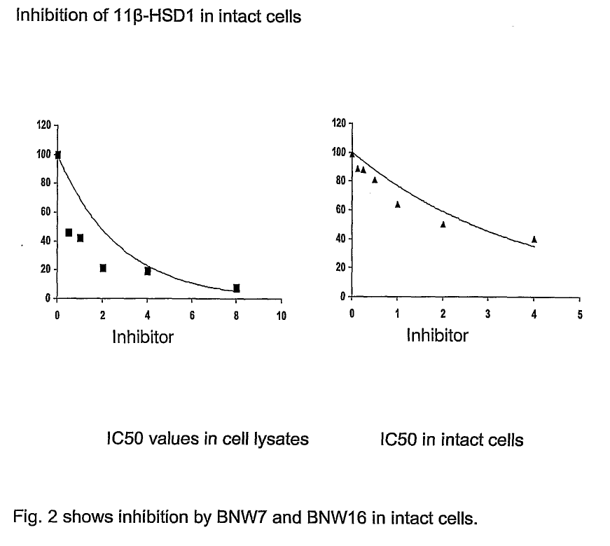

FIG. 2 shows inhibition of 11β-HSD1 by substance 7 (BNW7) and substance 16 (BNW16) in intact cells.

Preferred inhibitors 11β-HSD

| | |
|---|---|
| Standard: glycyrrhetinic acid | 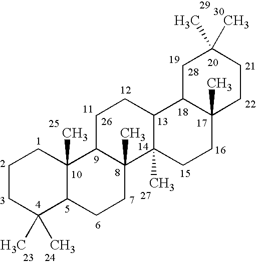 |
| | 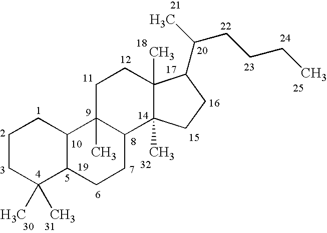 |
| Substance 7 | 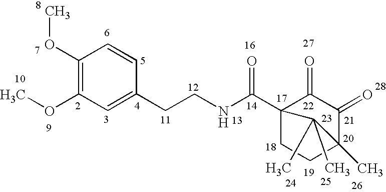 |
| | AutoNom Name: |
| | 4,7,7-Trimethyl-2,3-dioxo-bicyclo[2.2.1] |
| | heptane-1-carboxylic acid [2-(3, |
| | 4-dimethoxy-phenyl)-ethyl]-amide |
| Substance 13 | 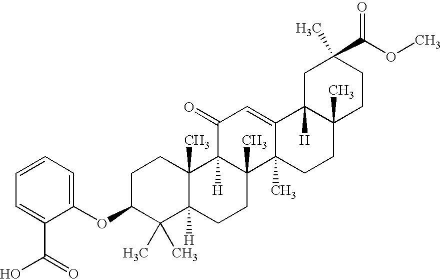 |
| | AutoNom Name: |
| | (2S,4aS,6aS,6bR,8aR,10S,12aS,12bR, |
| | 14bR)-10-(2-Carboxy-phenoxy)-2,4a,6a, |
| | 6b,9,9,12a-heptamethyl-13-oxo-1,2, |
| | 3,4,4a,5,6,6a,6b,7,8,8a,9,10,11,12, |
| | 12a,12b,13,14b-icosahydro-picene-2- |
| | carboxylic acid methyl ester |
| Substance 14 | 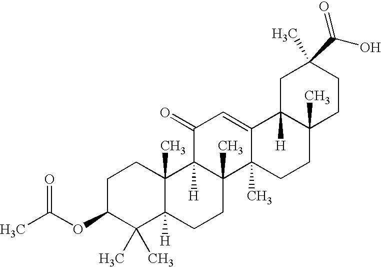 |
| | AutoNom Name: |
| | (2S,4aS,6aS,6bR,8aR,10S,12aS,12bR, |
| | 14bR)-10-Acetoxy-2,4a,6a,6b,9,9,12a- |
| | heptamethyl-13-oxo-1,2,3,4,4a,5,6,6a, |
| | 6b,7,8,8a,9,10,11,12,12a,12b,13, |
| | 14b-icosahydro-picene-2-carboxylic |
| | acid |
| Substance 16 | 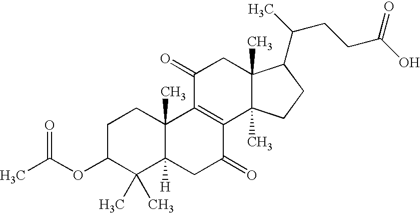 |
| | AutoNom Name: |
| | 4-((5R,10S,13R,14R)-3-Acetoxy-4,4, |
| | 10,13,14-pentamethyl-7,11-dioxo-2,3, |
| | 4,5,6,7,10,11,12,13,14,15,16,17- |
| | tetradecahydro-1H-cyclopenta[a] |
| | phenanthren-17-yl)-pentanoic acid |
| Substance 24 | 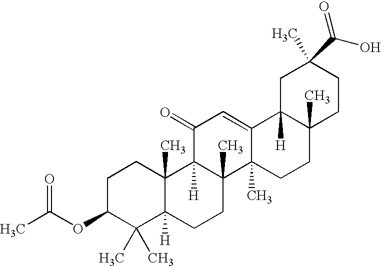 |
| | AutoNom Name: |
| | (2S,4aS,6aS,6bR,8aR,10S,12aS,12bR, |
| | 14bR)-10-Acetoxy-2,4a,6a,6b,9,9,12a- |
| | heptamethyt-13-oxo-1,2,3,4,4a,5,6, |
| | 6a,6b,7,8,8a,9,10,11,12,12a,12b,13, |
| | 14b-icosahydro-picene-2-carboxylic |
| | acid |
| Substance 25 | 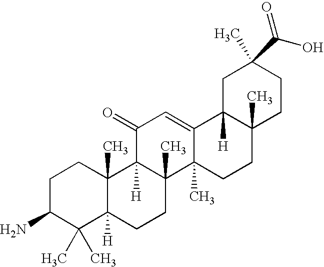 |
| | AutoNom Name: |
| | (2S,4aS,6aS,6bR,8aR,10S,12aS,12bR, |
| | 14bR)-10-Amino-2,4a,6a,6b,9,9,12a- |
| | heptamethyl-13-oxo-1,2,3,4,4a,5,6,6a, |
| | 6b,7,8,8a,9,10,11,12,12a,12b,13,14b- |
| | icosahydro-picene−2-carboxyiic acid |

### REFERENCES

1 Stewart, P. M. and Krozowski, Z. S. (1999) 11β-Hydroxysteroid dehydrogenase. Vitam. Horm. 57, 249-324
2 Lakshmi, V. and Monder, C. (1988) Purification and characterization of the corticosteroid 11β-dehydrogenase component of the rat liver 11β-hydroxysteroid dehydrogenase complex. Endocrinology 123, 2390-2398
3 Draper, N. et al. (2002) Association studies between microsatellite markers within the gene encoding human 11β-hydroxysteroid dehydrogenase type 1 and body mass index, waist to hip ratio, and glucocorticoid metabolism. J. Clin. Endocrinol. Metab. 87, 4984-4990
4 Bujalska, I. J. et al. (1997) Human 11β-hydroxysteroid dehydrogenase: studies on the stably transfected isoforms and localization of the type 2 isozyme within renal tissue. Steroids 77, 77-82
5 Agarwal, A. K. et al. (1990) Expression of 11β-hydroxysteroid dehydrogenase using recombinant vaccinia virus. Mol. Endocrinol. 4, 1827-1832
6 Walker, E. A. et al. (2001) Functional expression, characterization, and purification of the catalytic domain of human 11β-hydroxysteroid dehydrogenase type 1. J. Biol. Chem. 276, 21343-21350
7 Agarwal, A. K. et al. (1995) Gene structure and chromosomal localization of the human HSD11K gene encoding the kidney (type 2) isozyme of 11β-hydroxysteroid dehydrogenase. Genomics 29, 195-199
8 Stewart, P. M. et al. (1988) Syndrome of apparent mineralocorticoid excess: a defect in the cortisol-cortisone shuttle. J. Clin. Invest. 82, 340-349
9 Albiston, A. L. et al. (1994) Cloning and tissue distribution of the human 11β-hydroxysteroid dehydrogenase type 2 enzyme. Mol. Cell. Endocrinol. 105, R11-R17
10 Whorwood, C. B. et al. (1994) Epithelial cell localization of type 2 11β-hydroxysteroid dehydrogenase in rat and human colon. Endocrinology 135, 2533-2541
11 Stewart, P. M. et al. (1996) Hypertension in the syndrome of apparent mineralocorticoid excess due to mutation of the 11β-hydroxysteroid dehydrogenase type 2 gene. Lancet 347, 88-91
12 Mune, T. et al. (1995) Human hypertension caused by mutations in the kidney isozyme of 11β-hydroxysteroid dehydrogenase. Nat. Genet. 10, 394-399
13 Mune, T. and White, P. C. (1996) Apparent mineralocorticoid excess: genotype is correlated with biochemical phenotype. Hypertension 27, 1193-1199
14 Biason-Lauber, A. et al. (2000) Apparent cortisone reductase deficiency: a rare cause of hyperandrogenemia and hypercortisolism. Horm. Res. 53, 260-266
15 Jamieson, A. et al. (1999) Apparent cortisone reductase deficiency: a functional defect in 11β-hydroxysteroid dehydrogenase type 1. J. Clin. Endocrinol. Metab. 84, 3570-3574
16 Nordenstrom, A. et al. (1999) Failure of cortisone acetate treatment in congenital adrenal hyperplasia because of defective 11β-hydroxysteroid dehydrogenase reductase activity. J. Clin. Endocrinol. Metab. 84, 1210-1213
17 Phillipov, G. et al. (1996) Apparent cortisone reductase deficiency: a unique from of hypercortisolism. J. Clin. Endocrinol. Metab. 81, 3855-3860
18 Rodin, A. et al. (1994) Hyperandrogenism in polycystic ovary syndrome: evidence of dysregulation of 11β-hydroxysteroid dehydrogenase. N. Engl. J. Med. 330, 460-465
19 Condon, J. et al. (1998) Expression of type 2 11β-hydroxysteroid dehydrogenase and corticosteroid hormone receptors in early human fetal life. J. Clin. Endocrinol. Metab. 83, 4490-4497
20 Coulter, C. L. et al. (1998) Expression of 11β-HSD-2 in human adrenal cortical carcinoma and adenoma. Endocr. Res. 24, 875-876
21 Mune, T. et al. (2003) Role of local 11β-hydroxysteroid dehydrogenase type 2 expression in determining the phenotype of adrenal adenomas. J. Clin. Endocrinol. Metab. 88, 864-870
22 Korbonits, M. et al. (2001) Expression of 11β-hydroxysteroid dehydrogenase isoenzymes in the human pituitary: induction of the type 2 enzyme in corticotropinomas and other pituitary tumors. J. Clin. Endocrinol. Metab. 86, 2728-2733
23 Rabbitt, E. H. et al. (2003) Abnormal expression of 11β-hydroxysteroid dehydrogenase type 2 in human pituitary adenomas: a prereceptor determinant of pituitary cell proliferation. Oncogene 22, 1663-1667
24 Rabbitt, E. H. et al. (2002) Prereceptor regulation of glucocorticoid action by 11β-hydroxysteroid dehydrogenase: a novel determinant of cell proliferation. FASEB J. 16, 36-44
25 Sasano, H. et al. (1997) Localization of mineralocorticoid receptor and 11β-hydroxysteroid dehydrogenase type II in human breast and its disorders. Anticancer Res. 17, 2001-2007
26 Hundertmark, S. et al. (1997) Inhibition of 11β-hydroxysteroid dehydrogenase activity enhances the antiproliferative effect of glucocorticosteroids on MCF-7 and ZR-75-1 breast cancer cells. J. Endocrinol. 155, 171-180
27 Koyama, K. and Krozowski, Z. (2001) Modulation of 11β-hydroxysteroid dehydrogenase type 2 activity in Ishikawa cells is associated with changes in cellular proliferation. Mol. Cell. Endocrinol. 183, 165-170
28 Mirshahi, M. et al. (1997) Immunochemical demonstration of the mineralocorticoid receptor in ocular tissues. Neuroendocrinology 65, 70-78
29 Weinreb, R. N. et al. (1981) Detection of glucocorticoid receptors in cultured human trabecular cells. Invest. Opthalmol. Vis. Sci. 21, 403-407
30 David, R. et al. (1992) Diurnal intraocular pressure variations: an analysis of 690 diurnal curves. Br. J. Opthalmol. 76, 280-283
31 Starka, L. and Obenberger, J. (1976) Steroids and intraocular pressure. J. Steroid Biochem. 7, 979-983
32 Suzuki, T. et al. (2001) Immunohistochemical distribution of 11βhydroxysteroid dehydrogenase in human eye. Mol. Cell. Endocrinol. 173, 121-125
33 Stokes, J. et al. (2000) Distribution of glucocorticoid and mineralocorticoid receptors and 11β-hydroxysteroid dehydrogenases in human and rat ocular tissues. Invest. Opthalmol. Vis. Sci. 41, 1629-1638
34 Rauz, S. et al. (2001) Expression and putative role of 11β-hydroxysteroid dehydrogenase isozymes within the human eye. Invest. Opthalmol. Vis. Sci. 42, 2037-2042
35 Fraser, R. et al. (1999) Cortisol effects on body mass, blood pressure, and cholesterol in the general population. Hypertension 33, 1364-1368
36 Bujalska, I. J. et al. (1999) Differentiation of adipose stromal cells: the roles of glucocorticoids and 11β-hydroxysteroid dehydrogenase. Endocrinology 140, 3188-3196
37 Masuzaki, H. et al. (2001) A transgenic model of visceral obesity and the metabolic syndrome. Science 294, 2166-2170
38 Stewart, P. M. et al. (1999) Cortisol metabolism in human obesity: impaired cortisone-cortisol conversion in subjects with central adiposity. J. Clin. Endocrinol. Metab. 84, 1022-1027
39 Rask, E. et al. (2001) Tissue-specific dysregulation of cortisol metabolism in human obesity. J. Clin. Endocrinol. Metab. 86, 1418-1421
40 Rask, E. et al. (2002) Tissue-specific changes in peripheral cortisol metabolism in obese women: increased adipose 11β-hydroxysteroid dehydrogenase type 1 activity. J. Clin. Endocrinol. Metab. 87, 3330-3336
41 Livingstone, D. E. et al. (2000) Understanding the role of glucocorticoids in obesity: tissue-specific alterations of corticosterone metabolism in obese Zucker rats. Endocrinology 141, 560-563
42 Tomlinson, J. W. et al. (2002) Expression of 11β-hydroxysteroid dehydrogenase type 1 in adipose tissue is not increased in human obesity. J. Clin. Endocrinol. Metab. 87, 5630-5635
43 Walker, B. R. et al. (1995) Carbenoxolone increases hepatic insulin sensitivity in man: a novel role for 11β-oxosteroid reductase in enhancing glucocorticoid receptor activation. J. Clin. Endocrinol. Metab. 80, 3155-3159
44 Andrews, R. C. et al. (2003) Effects of the 11β-hydroxysteroid dehydrogenase inhibitor carbenoxolone on insulin sensitivity in men with type 2 diabetes. J. Clin. Endocrinol. Metab. 88, 285-291
45 Kotelevtsev, Y. et al. (1997) 11β-hydroxysteroid dehydrogenase type I knockout mice show attenuated glucocorticoid-inducible responses and resist hyperglycemia on obesity or stress. Proc. Natl. Acad. Sci. U.S.A. 94, 14924-14929
46 Morton, N. M. et al. (2001) Improved lipid and lipoprotein profile, hepatic insulin sensitivity, and glucose tolerance in 11β-hydroxysteroid dehydrogenase type 1 null mice. J. Biol. Chem. 276, 41293-41300
47 Barf, T. et al. (2002) Arylsulfonamidothiazoles as a new class of potential antidiabetic drugs. Discovery of potent and selective inhibitors of the 11β-hydroxysteroid dehydrogenase type 1. J. Med. Chem. 45, 3813-3815
48 Alberts, P. et al. (2002) Selective inhibition of 11β-hydroxysteroid dehydrogenase type 1 decreases blood glucose concentrations in hyperglycaemic mice. Diabetologia 45, 1528-1532
49 Canalis, E. (1996) Clinical review 83: mechanisms of glucocorticoid action in bone: implications to glucocorticoid-induced osteoporosis. J. Clin. Endocrinol. Metab. 81, 3441-3447
50 Bland, R. et al. (1999) Characterization of 11β-hydroxysteroid dehydrogenase activity and corticosteroid receptor expression in human osteosarcoma cell lines. J. Endocrinol. 161, 455-464
51 Eyre, L. J. et al. (2001) Expression of 11β-hydroxysteroid dehydrogenase in rat osteoblastic cells: pre-receptor regulation of glucocorticoid responses in bone. J. Cell. Biochem. 81, 453-462
52 Cooper, M. S. et al. (2000) Expression and functional consequences of 11β-hydroxysteroid dehydrogenase activity in human bone. Bone 27, 375-381
53 Cooper, M. S. et al. (2001) Modulation of 11β-hydroxysteroid dehydrogenase isozymes by proinflammatory cytokines in osteoblasts: an autocrine switch from glucocorticoid inactivation to activation. J. Bone Miner. Res. 16, 1037-1044
54 Cooper, M. S. et al. (2002) Osteoblastic 11β-hydroxysteroid dehydrogenase type I activity increases with age and glucocorticoid exposure. J. Bone Miner. Res. 17, 979-986
55 Bengtsson, B. A. et al. (1989) Body composition in acromegaly. Clin. Endocrinol. 31, 481-490
56 Sacca, L. et al. (1994) Growth hormone and the heart. Endocr. Rev. 15, 555-573
57 de Boer, H. et al. (1995) Clinical aspects of growth hormone deficiency in adults. Endocr. Rev. 16, 63-86
58 Gelding, S. V. et al. (1998) The effect of growth hormone replacement therapy on cortisol-cortisone interconversion in hypopituitary adults: evidence for growth hormone modulation of extrarenal 11β-hydroxysteroid dehydrogenase activity. Clin. Endocrinol. 48, 153-162
59 Moore, J. S. et al. (1999) Modulation of 11β-hydroxysteroid dehydrogenase isozymes by growth hormone and insulin-like growth factor: in vivo and In vitro studies. J. Clin. Endocrinol. Metab. 84, 4172-4177
60 Trainer, P. J. et al. (2001) Modulation of cortisol metabolism by the growth hormone receptor antagonist pegvisomant in patients with acromegaly. J. Clin. Endocrinol. Metab. 86, 2989-2992
61 Schweizer, R. A., Atanasov, A. G., Frey, B. M., and Odermatt, A. (2003) Mol Cell Endocrinol 212, 41-49.
62 Christoph Frick, Atanas G. Atanasov, Peter Arnold, Juris Ozols, and Alex Odermatt (2004) J Biol Chem, 279, 131-138.

1. Use of an 11β-HSD inhibitor or a pharmaceutically acceptable salt thereof for the manufacture of a pharmaceutical agent for the prevention and/or treatment of metabolic diseases.

2. Use according to claim 1, wherein the inhibitor is selected from the group consisting of substances 16, 7, 13, 14, 24, 25, 1-6, 8-12, 15, 17-23 or a compound of formula I, II or III.

3. Use according to claim 1 for the manufacture of a pharmaceutical agent for the prevention and/or treatment of obesity or insulin sensitivity.

4. Use according to claim 1 for the manufacture of a pharmaceutical agent for the prevention and/or treatment of diabetes type 11.

5. Use of an 11β-HSD inhibitor or a pharmaceutically acceptable salt thereof for the manufacture of a pharmaceutical agent for the prevention and/or treatment of cancer and/or cell proliferation.

6. Use according to claim 5, wherein the inhibitor is selected from the group consisting of substances 16, 7, 13, 14, 24, 25, 1-6, 8-12, 15, 17-23 or a compound of formula I, II or III.

7. Use according to claim 5 for the manufacture of a pharmaceutical agent for the prevention and/or treatment of breast cancer.

8. Use of an 11β-HSD inhibitor or a pharmaceutically acceptable salt thereof for the manufacture of a pharmaceutical agent for the prevention and/or treatment of glaucoma.

9. Use according to claim 8, wherein the inhibitor is selected from the group consisting of substances 16, 7, 13, 14, 24, 25, 1-6, 8-12, 15, 17-23 or a compound of formula I, II or III.

10. Use of an 11β-HSD inhibitor or a pharmaceutically acceptable salt thereof for the manufacture of a pharmaceutical agent for the prevention and/or treatment of diseases associated with abnormal growth hormone secretion or for the manufacture of a pharmaceutical agent for the prevention and/or treatment of wound healing disorders.

11. Use according to claim 10, wherein the inhibitor is selected from the group consisting of substances 16, 7, 13, 14, 24, 25, 1-6, 8-12, 15, 17-23 or a compound of formula I, II or III.

12. Use according to claim 1, wherein the 11β-HSD inhibitor is selected from substances 16, 7, 13, 24 or 25.

13. A pharmaceutical composition comprising, as an active ingredient, an 11β-HSD inhibitor or a salt thereof, wherein said 11β-HSD type 1 inhibitor is selected from the group consisting of the following formulas I to III: 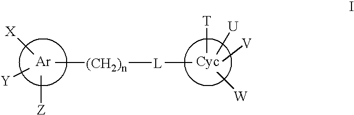
wherein
X, Y and Z each independently represent halogen, in particular, F, Cl, I or Br, C₁-C₆ alkyl, C₅-C₅ aryl or C₁-C₆ alkoxy,
n represents an integer from 1 to 10, in particular, from 1 to 4,
L represents an amide, amine, sulfonamide, ester, thioester or keto group,
T, U, V and W each independently represent an oxo, thio, ketone, thioketone, C₁-C₆ alkyl or C₁-C₆ alkanol group,
Ar represents an aromatic ring system, and
Cyc represents a cyclic ring system,
with the proviso that the compound is not substance 7, 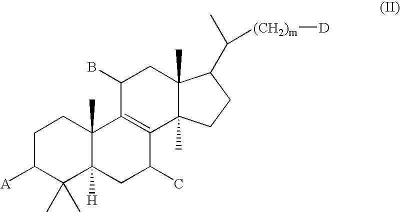
wherein
A represents an OH, a C₁-C₁₀ ester (C₁-C₁₀ alkyl-CO—O—), a C₁-C₁₀ amide (C₁-C₁₀ alkyl-CO—NH—), a C₁-C₁₀ ether or a C₁-C₁₀ ketone (C₁-C₁₀ alkyl-CO—) group,
B and C each independently represent an oxo group, a keto group, a C₁-C₆ alkanol group or a C₁-C₆ alkyl group,
m is an integer from 1 to 10, in particular, from 1 to 4, and
D is a group selected from COOR¹ or CONR²R³, wherein R¹, R² and R³ each independently represent H or a C₁-C₆ alkyl group,
with the proviso that the compound is not substance 16, 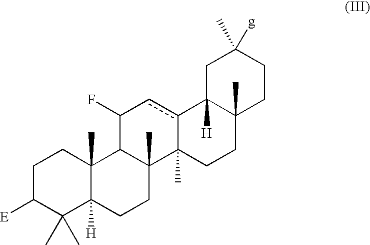
wherein
E represents —OH, a C₁-C₁₀ ester (C₁-C₁₀ alkyl-CO—O—), a C₁-C₁₀ amide (C₁-C₁₀ alkyl-CO—NH—), a C₁-C₁₀ ether or a C₁-C₁₀ ketone (C₁-C₁₀ alkyl-CO—) group,
F represents an oxo group, a keto group, a C₁-C₆ alkanol group or a C₁-C₆ alkyl group,
and
G is a group selected from COOR¹ or CONR²R³, wherein R¹, R² and R³ each independently represent H or a C₁-C₂₀ hydrocarbon group, in particular, a C₁-C₆ alkyl group,
with the proviso that the compound is not a compound of substance 24 or 25.

14. A method of preventing and/or treating a metabolic disease in a patient in need of such prevention or treatment, the method comprising administering to the patient an effective amount of an 11β-HSD inhibitor or a pharmaceutically acceptable salt thereof.
